(19) United States
(12) Patent Application Publication
(10) Pub. No.: US 2008/0287540 A1
(43) Pub. Date: Nov. 20, 2008

(54) LIP CARE COMPOSITIONS

(75) Inventors: Allison Wright, Bethel, CT (US); Richard Loren McManus, Shelton, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 11/749,797

(22) Filed: May 17, 2007

(51) Int. Cl. A61K 31/20; A61Q 17/00

(52) U.S. Cl. 514/558; 514/738

(57) ABSTRACT

A lip care composition is provided which includes at least 40% petrolatum, at least 5% glycerin, and 1 to 20% of a C₃ (alkyl or hydroxyalkyl) alcohol ester of isostearic acid, wherein the glycerin and the isostearic acid ester are present in a weight ratio ranging from 5:1 to 1:1. A method for moisturization of lips is also provided through application of the composition to the lips.

### BACKGROUND OF THE INVENTION

1. Field of the Invention

The invention relates to cosmetic compositions particularly applied to human lips, for depositing thereon a protective and moisturizing coating.

2. The Related Art

Dry and cold environments cause chapping and cracking of the lips. Salves in stick or paste form have provided protection against the environmental onslaught. These materials generally consist of an emollient base capable of spreading easily to form a thin protective layer.

A number of commercial products are available. The majority of the products are based on occlusives and structurants, particularly petrolatum and waxes. Illustrative is Vaseline® Lip Therapy™ which essentially is 100% white petrolatum with minor levels of flavor and sweetener. Some lip balms such as Blistex® Complete Moisture and Blistex® ProRelief contain up to 1.4% glycerin.

U.S. Pat. No. 5,085,856 (Dunphy et al.) reports a moisturizing lipstick which is an oil and water emulsion. Besides up to 20% water, the sticks are exemplified with 5% glycerin (glycerol) in the aqueous phase.

While there have been advances in the treatment of chapped lips, the problems have not been fully solved. It is an object of the present invention to provide a lip treatment composition which imparts superior moisturization as an immediate effect and keeps working over an extended period of time.

### SUMMARY OF THE INVENTION

A lip care composition is provided which includes:
(i) from 40 to 90% by weight of petrolatum;
(ii) from 5 to 40% by weight of glycerin;
(iii) from 1 to 20% by weight of a C₃ (alkyl or hydroxyalkyl) alcohol ester of isostearic acid; and
wherein glycerin and the isostearic acid ester are present in a weight ratio ranging from 5:1 to 1:1.

Further, a method for improving moisturization is provided which involves applying the lip care composition to the lips. Advantageously, the application will result in a SkiCon value of at least 20% hydration at 8 hours compared to baseline; a Corneometer value of at least 1.0 at 8 hours compared to baseline; and an Elasticity improvement of at least 3% at 8 hours compared to baseline.

### DETAILED DESCRIPTION OF THE INVENTION

Now it has been found that superior immediate and long-lasting moisturization of lips can be achieved with a combination of petrolatum, glycerin and a C₃ (alkyl or hydroxyalkyl) alcohol ester of isostearic acid. Besides a high level of occlusive substance (petrolatum), formulas of the present invention incorporate relatively high levels of humectant (glycerin) In particular, it has been discovered that a C₃ (alkyl or hydroxyalkyl) alcohol ester of isostearic acid synergistically with glycerin adds to the humectancy properties.

Petrolatum known also as petroleum jelly is a purified mixture of semi-solid hydrocarbons, chiefly of the methane series of the general formula CₙH₂ₙ₊₂ from petroleum. More specifically, petrolatum is a colloidal system of non-straight-chain solid hydrocarbons and high-boiling liquid hydrocarbons, in which most of the liquid hydrocarbons are held inside micelles. It also has been described as a yellowish to light amber or white, semi-solid, unctuous mass; practically odorless and tasteless. Density at 25° C. is reported as 0.820-0.865, melting point 38-54° C. and refractive index of 1.460 to 1.474. According to the present invention, petrolatum is used at a level from 40 to 90%, preferably from 45 to 80%, and optimally from 48 to 60% by weight of the composition.

Glycerin which is also known as glycerol is a second important component of compositions according to the present invention. Glycerin will be present at levels from 5 to 40%, preferably from 8 to 30%, even more preferably from 10 to 25%, and optimally from 12 to 20% by weight of the composition.

A third component of compositions according to the present invention is a C₃ (alkyl or hydroxyalkyl) alcohol ester of isostearic acid (hereinafter the “estee”). Particularly effective esters are isopropyl isostearate and glyceryl isostearate. The ester may have a molecular weight ranging from about 325 to about 360. Levels of the ester may range from 1 to 20%, preferably from 3 to 18%, more preferably from 5 to 15% by weight of the composition. Desirably, glycerin and the ester may be present in a weight ratio ranging from 5:1 to 1:1, preferably from 3:1 to 1:1, and optimally from 2:1 to 1.2:1, most especially 1.5:1.

Compositions of the present invention may contain a variety of other components for imparting certain functional attributes. Generally these are optional components and they must be suitable for use in contact with the lips. Components must be without toxicity to the body, have compatibility, physical stability and be non-allergenic.

Although the preferred compositions may contain essentially no water (i.e. less than 0.3% water), some formulations may contain from about 0.5 to about 20%, possibly from about 1 to about 10%, and very possibly from about 1.5 to about 5% water by weight of the composition.

Waxes may be incorporated into compositions of the present invention. These are particularly important for embodiments wherein the lip care composition is in a stick form. These materials may typically be incorporated at levels from about 0.1 to about 20%, sometimes from about 0.5 to about 10% (e.g. stick formulations), and in other formulations (e.g. tin and tube packaging) from 0% to about 2% by weight of the composition.

Waxes are defined as low-melting organic mixtures or compounds of high molecular weight, solid at room temperature and generally similar in composition to fats and oils except that they contain no glycerides. Some are hydrocarbons, others are esters of fatty acids and alcohols. Waxes useful in the present invention are selected from the group consisting of animal waxes, vegetable waxes, mineral waxes, synthetic waxes, petroleum waxes, ethylenic polymers, hydrocarbon types such as Fischer-Tropsch waxes, silicone waxes, and mixtures thereof wherein the waxes have a melting point between 55° C. and 100° C. and a needle penetration, as measured according to the American standard ASTM D5, of 3 to 40 at 25° C.

The specific waxes which may be useful can be selected from the group consisting of beeswax, lanolin wax, shellac wax (animal waxes); carnauba, candelilla, bayberry (vegetable waxes); ozokerite, ceresin (mineral waxes); paraffin, microcrystalline waxes (petroleum waxes); polyethylene (ethylenic polymers); polyethylene homopolymers (Fischer-Tropsch waxes); C24-45 alkyl methicones (silicone waxes); and mixtures thereof. Most preferred are beeswax, lanolin wax, carnauba, candelilla, ozokerite, ceresin, paraffins, microcrystalline waxes, polyethylene, C24-45 alkyl methicones, and mixtures thereof.

Fats are glyceryl esters of higher fatty acids such as stearic and palmitic. Such esters and their mixtures are solid at room temperature and exhibit crystalline structure. Fats are typically used at levels from about 5% to about 50%, preferably from about 10% to about 25%, and most preferably from about 10% to about 20% by weight of the composition.

The fats employed according to the invention are selected from the group consisting of fats derived from animals, vegetables, synthetically derived fats, and mixtures having a melting point from about 55° C. to about 100° C. and a needle penetration, as measured according to ASTM D5, from about 3 to about 40 at 25° C. Preferably, the fats are selected from the group consisting of glyceryl monostearate, glyceryl distearate, glyceryl tristearate, palmitate esters of glycerol, C18-36 triglycerides, glyceryl tribehenate, C18-36 acid triglycerides and mixtures thereof.

Hydrophobic conditioning agents may be included which can be selected from mineral oil, lecithin, hydrogenated lecithin, lanolin, lanolin derivatives, C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, polydialkylsiloxanes, polydiarylsiloxanes, polyalkarylsiloxanes, cyclomethicones having 3 to 9 silicon atoms, vegetable oils, hydrogenated vegetable oils, polypropylene glycol C4-C20 alkyl ethers, di C8-C30 alkyl ethers, and combinations thereof.

Emulsifiers may be useful, especially in the presence of hydrophilic components. Levels of emulsifier may range from about 0.1 to about 10%, preferably from about 0.3 to about 5% by weight of the composition. Particularly useful are phospholipids such as lecithin and also glycerol fatty acid esters such as glycerol monostearate,

Certain embodiments may require polymers and copolymers to structure the compositions. Levels of these materials may range from about 0.1 to about 10%, preferably from about 0.5 to about 8%, more preferably from about 1 to about 5% by weight of the composition.

Illustrative are polyvinyl pyrrolidone (PVP) polymers and copolymers.

The copolymers are formed with alpha-olefins. Illustrative are vinyl pyrrolidone/Eicosene copolymer and vinyl pyrrolidone/Hexadecene, commercially available as Ganex 220 and Ganex 216 respectively from ISP Corporation.

The present compositions may comprise vitamin compounds, precursors, and derivatives thereof. These vitamin compounds may be in either natural or synthetic form. Suitable vitamin compounds include, but are not limited to, Vitamin A (e.g. beta carotene, retinoic acid, retinol, retinoids, retinyl paimitate, retinyl proprionate), Vitamin B (e.g. niacin, niacinamide, riboflavin, pantothenic acid), Vitamin C (e.g. ascorbic acid), Vitamin D (e.g. ergosterol, ergocalciferol, cholecalciferol), Vitamin E (e.g. tocopherol, tocopherol acetate), and Vitamin K (e.g., phytonadione, menadione, phthiocol), compounds. Amounts of the vitamins may range from 0.000001 to 2% by weight of the composition.

Sunscreens may be incorporated into the lip care compositions of this invention. Levels of sunscreen may range from about 0.1 to about 10%, preferably from about 1 to about 3% by weight of the composition.

Nonlimiting examples of sunscreens are 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyidibenzoylmethane, 4-isopropyl dibenzoylmethane, 3-benzylidene camphor, 3-(4-methylbenzylidene)camphor, titanium dioxide, zinc oxide, silica, iron oxide, and mixtures thereof. Particularly preferred are sunscreens commercially available under the trademarks Parsol® MCX, Parsol® 1789 and Mexoryl®.

The compositions of the present invention may also include a flavanoid compound. Flavanoids are broadly disclosed in U.S. Pat. Nos. 5,686,082 and 5,686,367 herein incorporated by reference.

Flavonoids suitable for use in the present invention are flavanones selected from the group consisting of unsubstituted flavanones, mono-substituted flavanones, and mixtures thereof; chalcones selected from the group consisting of unsubstituted chalcones, mono-substituted chalcones, di-substituted chalcones, tri-substituted chalcones, and mixtures thereof; flavones selected from the group consisting of unsubstituted flavones, mono-substituted flavones, di-substituted flavones, and mixtures thereof; one or more isoflavones; coumarins selected from the group consisting of unsubstituted coumarins, mono-substituted coumarins, di-substituted coumarins, and mixtures thereof; chromones selected from the group consisting of unsubstituted chromones, mono-substituted chromones, di-substituted chromones, and mixtures thereof; one or more dicoumarols; one or more chromanones; one or more chromanols; isomers (e.g. cis/trans isomers) thereof; and mixtures thereof. By the term “substituted” as used herein means flavanoids wherein one or more hydrogen atoms of the flavanoid has been independently replaced with hydroxyl, C1-C8 alkyl, C1-C4 alkoxyl, O-glycoside, and a mixture of these substituents.

Preferred for use herein are unsubstituted flavanone, methoxy flavanones, unsubstituted chalcone, 2′,4-dihydroxychalcone, and mixtures thereof. Most preferred are unsubstituted flavanone, unsubstituted chalcone (especially the trans isomer), and mixtures thereof

Sterols may also be included in the presently claimed compositions. Examples of useful sterol compounds include sitosterol, stigmasterol, campesterol, brassicasterol, lanosterol, 7-dehydrocholesterol, and mixtures thereof. These can be synthetic in origin or from natural sources, e.g., blends extracted from plant sources (e.g., phytosterols).

Flavors and herbal extracts may also be incorporated into the lip care compositions. The herbal extracts may either be water soluble or water-insoluble carried in a solvent which respectively is hydrophilic or hydrophobic. Water and ethanol are the preferred extract solvents. Illustrative extracts include those from green tea, chamomile, licorice, aloe vera, grape seed, citrus unshui, willow bark, sage, thyme and rosemary.

Also included may be such materials as lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides, sphingolipids and phytosphingolipids may also be useful. Amounts of these materials may range from about 0.000001 to about 10%, preferably from about 0.0001 to about 1% by weight of the composition.

Preferably some compositions of the present invention will not contain any colorants such as dyes or pigments, except for titanium dioxide. Iron oxides as well as dyes or other pigments are preferably absent or at least present at no higher than 0.1%, preferably no higher than 0.001%, and particularly from 0 to 0,00001% by weight.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

All documents referred to herein, including all patents, patent applications, and printed publications, are hereby incorporated by reference in their entirety in this disclosure.

It should be noted that in specifying any range of concentration or amount, any particular upper concentration can be associated with any particular lower concentration or amount.

The following examples will more fully illustrate the embodiments of this invention. All parts, percentages and proportions referred to herein and in the appended claims are by weight unless otherwise illustrated.

### EXAMPLE 1

A clinical moisturization study was conducted on several compositions to establish relative efficacy. The base formula was a lip balm whose constituents are outlined under Table I.

TABLE I
Base Formula

| COMPONENT | WEIGHT % |
|---|---|
| Petrolatum | * |
| Glycerin | * |
| Isopropyl Isostearate | * |
| Paraffin Wax | 14.4 |
| Prolipid ® 151** | 5.0 |
| Vinyl Pyrrolidone/Eicosene Copolymer | 1.0 |
| Di-PPG-3 Myristyl Ether Adipate | 1.0 |
| Shea Butter | 1.0 |
| Microcrystalline Wax | 0.15 |
| Water | 5.0 |
| Weight % | 100% |

*See Table II for percentages.
**INCI name: Glyceryl Stearate (and) Cetyl Alcohol (and) Stearyl Alcohol (and) Behenyl Alcohol (and) Palmitic Acid (and) Stearic Acid.

The base formula was used to prepare Samples A-D formulated with different amounts of glycerin, petrolatum and isopropyl isostearate. See Table II below.

TABLE II

Weight %

| | | | Isopropyl |
|---|---|---|---|
| Sample | Petrolatum | Glycerin | Isostearate |
| A | 47.45 | 15.0 | 10.0 |
| B | 52.45 | 10.0 | 10.0 |
| C | 57.45 | 5.0 | 10.0 |
| D | 52.45 | 15.0 | 5.0 |
| Chapstick ® | 38+ | 0 | 0 |
| Lip Balm | | | |
| Blistex ® | — | 1.43 | 0 |
| Complete | | | |
| Moisture | | | |

Seventeen panelists were recruited for the clinical study. Panelists were asked to stop shaving and using moisturizers on their legs two days before the study. Panelists were not to wet their legs at least two hours before the study. Baseline values were taken on panelist skin for each of the test procedures. A sample of 0.05 gram experimental sample was then applied onto a 5×5 cm area marked on each outer calf. Instrumental measurements were taken at 4 hours and 8 hours post product application.

The SkiCon 200 instrument was used for analysis. Moisturization in this test is measured on a skin surface through a conductance evaluation (micro Siemens). Depth of measurement is approximately less than 15 μm. Results are reported as area measured under a curve graphing the % Hydration versus time. Results are found in Table III.

TABLE III
SkiCon Results

| Sample | Hydration (%) at 8 Hours |
|---|---|
| A | 26.53 |
| B | 15.80 |
| C | 18.08 |
| D | 19.02 |
| Chapstick ® Lip Balm | 14.12 |
| Blistex ® Complete Moisture | 2.22 |

Measurement was also taken on a Corneometer. Results are recorded in Table IV.

TABLE IV
Corneometer Results

| Sample | Hydration (%) at 8 Hours |
|---|---|
| A | 3.15 |
| B | 0.48 |
| C | −0.15 |
| D | −1.26 |
| Chapstick ® Lip Balm | −3.70 |
| Blistex ® Complete Moisture | −1.81 |

Elasticity is a measure of the elastic recovery that occurs immediately upon full release of negative pressure. Skin elasticity changes from the baseline at 8 hours was measured with a BTC 2000 Instrument. Results are recorded in Table V.

TABLE V
Elasticity Change

| Sample | Elasticity (%) at 8 Hours |
|---|---|
| A | 5.14 |
| B | 0.89 |
| C | −4.89 |
| D | −2.6 |
| Chapstick ® Lip Balm | 1.83 |
| Blistex ® Complete Moisture | −7.09 |

Sample A performed best. It exhibited the highest moisturization value in both the SkiCon and Corneometer evaluation methods. It appears that a high level of glycerin is responsible for significant improvement over the commercial lip balms. Of particular interest, is that Sample A performed significantly better than Sample D. This indicates that improvement in moisturization may result from interaction of glycerin and isopropyl isostearate.

Based on the BTC 2000 data, it is also seen that Sample A had a significantly higher elasticity value than all the other experimental Samples, including Sample D.

### EXAMPLE 2

Skin hydration was further evaluated on a group of 16 panelists utilizing a non-contact method known as Near Infrared (NIR) imaging system. The materials tested were Sample A outlined in Tables I and II and the Chapsticks and Blistex commercial products. Additionally tested were Samples E, F and G. These Samples had formulas identical to Sample A except that the isopropyl isostearate was replaced with an equivalent 10% amount of isopropyl myristate, isopropyl palmitate and glyceryl isostearate, respectively.

NIR testing was conducted in a temperature humidity controlled room (20° C. temperature and 40% humidity). The imaging system consisted of a near infrared camera, two halogen lights, and six optical near infrared filters at the following spectra: 1280, 1390, 1410, 1430, 1450, 1470, and 1620 cm⁻¹. Water spectral absorption was resolved in this near infrared region. The evaluation was done at baseline, 2 hours and 4 hours. Area under the curve (AUC) analysis used for this study is defined as:

AUC=((m2+m1)/2×t2−t1)+((m3+m2)/2×t3−t2)

where

m1=NIR reading at 00

m2=NIR reading at 120 minutes

m3=NIR reading at 240 minutes

t1=0

t2=120 minutes

t3=240 minutes

Results of these experiments are reported in Table VI.

TABLE VI
NIR Results

| Sample | Ester | AUC |
|---|---|---|
| A | Isopropyl Isostearate | 765.86 |
| E | Isopropyl Myristate | 727.74 |
| F | Isopropyl Palmitate | 733.65 |
| G | Glyceryl Isostearate | 769.24 |
| Chapstick ® Lip Balm | | 734.52 |
| Blistex ® Complete Moisture | | 729.57 |

Higher value for the AUC corresponds to an improved skin hydration effect. From the experiments reported in Table VI, it is demonstrated that isopropyl myristate and isopropyl palmitate were inferior to identical formulations substituted with isopropyl isostearate and glyceryl isostearate, respectively. Samples A and G were also shown to be better at skin hydration than the commercial Chapstick® and Blistex® products

1. A lip care composition comprising:
(i) from 40 to 90% by weight of petrolatum;
(ii) from 5 to 40% by weight of glycerin;
(iii) from 1 to 20% by weight of isopropyl isostearate; and
wherein glycerin and the isopropyl isostearate are present in a respective weight ratio ranging from about 2:1to about 2:1.

2-3. (canceled)

4. The composition according to claim 1 wherein the ratio of glycerin to isopropyl isostearate is 1.5:1.

5. The composition according to claim 1 exhibiting a SkiCon value of at least 2% hydration at 8 hours relative to baseline.

6. The composition according to claim 1 exhibiting a Corneometer value of at least 1.0 at 8 hours relative to baseline.

7. The composition according to claim 1 wherein elasticity increases at least 30% relative to baseline after 8 hours.

8. The composition according to claim 1 wherein petrolatum is present from 45 to 80% by weight of the composition.

9. The composition according to claim 1 wherein glycerin is present from 10 to 25% by weight of the composition.

10-11. (canceled)

12. A method for improving moisturization of the lips comprising applying to the lips a composition comprising:
(i) from 40 to 90% by weight of petrolatum;
(ii) from 5 to 40% a by weight of glycerin;
(iii) from 1 to 20% a by weight of a C₃ (alkyl or hydroxyalkyl) alcohol ester of isostearic acid; and
wherein glycerin and the isostearic acid ester are present in a weight ratio ranging from 5:1 to 1:1.

13. A lip care composition comprising:
(i) from 40 to 90% by weight of petrolatum;
(ii) from 5 to 40% by weight of glycerin;
(iii) from 1 to 20% by weight of glyceryl isostearate; and
wherein glycerin and glyceryl isostearate are present in a respective weight ratio ranging from about 2:1 to about 1.2:1.

14. The composition according to claim 13 wherein the ratio of glycerin to glycerol isostearate is 1.5:1.
